(19) United States
(12) Patent Application Publication
(10) Pub. No.: US 2021/0035661 A1
(43) Pub. Date: Feb. 4, 2021

(54) METHODS AND SYSTEMS FOR RELATING USER INPUTS TO ANTIDOTE LABELS USING ARTIFICIAL INTELLIGENCE

(71) Applicant: KPN Innovations, LLC, Lakewood, CO (US)

(72) Inventor: Kenneth Neumann, Lakewood, CO (US)

(21) Appl. No.: 16/529,852

(22) Filed: Aug. 2, 2019

(51) Int. Cl. G16H 10/40; G06K 9/62; G16H 10/60

(52) U.S. Cl. CPC G16H 10/40; G16H 10/60; G06K 9/6218

(57) ABSTRACT

A system for relating user inputs to antidote labels using artificial intelligence. The system includes at least a server designed and configured to receive at least a user input datum. The at least a server is designed and configured to create at least an unsupervised machine-learning model as a function of the at least a user input datum and output at least a first proving element. The at least a server is configured to select at least a first training set as a function of the at least a user input datum and the at least a first probing element. The system includes at least a label learner operating on the at least a server configured to create at least a supervised machine-learning model using the at least a first training set and relate at least a user input datum to at least an antidote. At least a label learner is configured to generate at least an antidote output using the at least a user input datum and the at least a supervised machine-learning model.

### FIELD OF THE INVENTION

The present invention generally relates to the field of artificial intelligence. In particular, the present invention is directed to methods and systems for relating user inputs to antidote labels using artificial intelligence.

### BACKGROUND

Accurate selection and analysis of data can be challenging, due to the multitude of data and factors to consider. Inaccurate and incorrect data selection can lead to unfavorable outcomes. Ensuring proper selection and utilization is of utmost importance.

### SUMMARY OF THE DISCLOSURE

In an aspect, a system for relating user inputs to antidote labels using artificial intelligence. The system comprising at least a server. The at least a server designed and configured to receive at least a user input datum wherein the at least a user input datum further comprises at least a user structure entry. The at least a server designed and configured to create at least an unsupervised machine learning model as a function of the at least a user input datum wherein creating at least an unsupervised machine learning model further comprises selecting at least a dataset as a function of the at least a user structure entry wherein the at least a dataset further comprises at least a datum of structure entry data and at least a correlated antidote element; and generating at least an unsupervised machine-learning model wherein generating the at least an unsupervised machine-learning model further comprises generating at least a clustering model to output at least a probing element containing at least a commonality label as a function of the at least a user structure entry and the at least a dataset. The at least a server designed and configured to select at least a first training set as a function of the at least a user structure entry and the at least a first probing element containing the at least a commonality label. The system includes at least a label learner operating on the at least a server; the at least a label learner designed and configured to create at least a supervised machine learning model as a function of the at least a first training set and the at least a commonality label, wherein creating the at least a supervised machine learning model further comprises generating at least a supervised machine-learning model to output at least an antidote output as a function of relating the at least a user input datum to at least an antidote.

In an aspect, a method of relating user inputs to antidote labels using artificial intelligence. The method includes receiving by at least a server at least a user input datum wherein the at least a user input datum further comprises at least a user structure entry. The method includes creating by the at least a server at least an unsupervised machine learning model as a function of the at least a user input datum wherein creating at least an unsupervised machine learning model further comprises selecting at least a dataset as a function of the at least a user structure entry wherein the at least a dataset further comprises at least a datum of structure entry data and at least a correlated antidote element; and generating at least an unsupervised machine-learning model wherein generating the at least an unsupervised machine-learning model further comprises generating at least a clustering model to output at least a probing element containing at least a commonality label as a function of the at least a user structure entry and the at least a dataset. The method includes selecting by the at least a server at least a first training set as a function of the at least a user structure entry and the at least a first probing element containing the at least a commonality label. The method includes creating by at least a label learner operating on the at least a server at least a server at least a supervised machine learning model as a function of the at least a first training set and the at least a commonality label, wherein creating the at least a supervised machine learning model further comprises generating at least a supervised machine-learning model to output at least an antidote output as a function of relating the at least a user input datum to at least an antidote.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is a block diagram illustrating an exemplary embodiment of a system for relating user inputs to antidote labels using artificial intelligence;

FIG. 2 is a block diagram illustrating an exemplary embodiment of an unsupervised learning module;

FIG. 3 is a block diagram illustrating an exemplary embodiment of an unsupervised database;

FIG. 4 is a block diagram illustrating an exemplary embodiment of an expert knowledge database;

FIG. 5 is a block diagram illustrating an exemplary embodiment of a training set database;

FIG. 6 is a block diagram illustrating an exemplary embodiment of a label learner;

FIG. 7 is a block diagram illustrating an exemplary embodiment of a variables database;

FIG. 8 is a flow diagram illustrating an exemplary embodiment of a method of relating user inputs to antidote labels using artificial intelligence; and

FIG. 9 is a block diagram of a computing system that can be used to implement any one or more of the methodologies disclosed herein and any one or more portions thereof.

The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

At a high level, aspects of the present disclosure are directed to systems and methods for relating user inputs to antidote labels using artificial intelligence. In an embodiment, at least a server receives at least a user input datum. User input datum may include a description of a symptom user may be experiencing or a tissue sample analysis of a user such as a blood test showing levels of commensal bacteria or a saliva test showing salivary levels of hormone levels of a user. At least a server creates at least an unsupervised machine-learning model as a function of the at least a user input datum and outputs at least a first probing element. First probing element may include clusters of data or groups of data that may be utilized to select at least a first training set. At least a server includes at least a label learner operating on the at least a server wherein the at least a label learner is configured to create at least a supervised machine-learning model using the at a first training set.

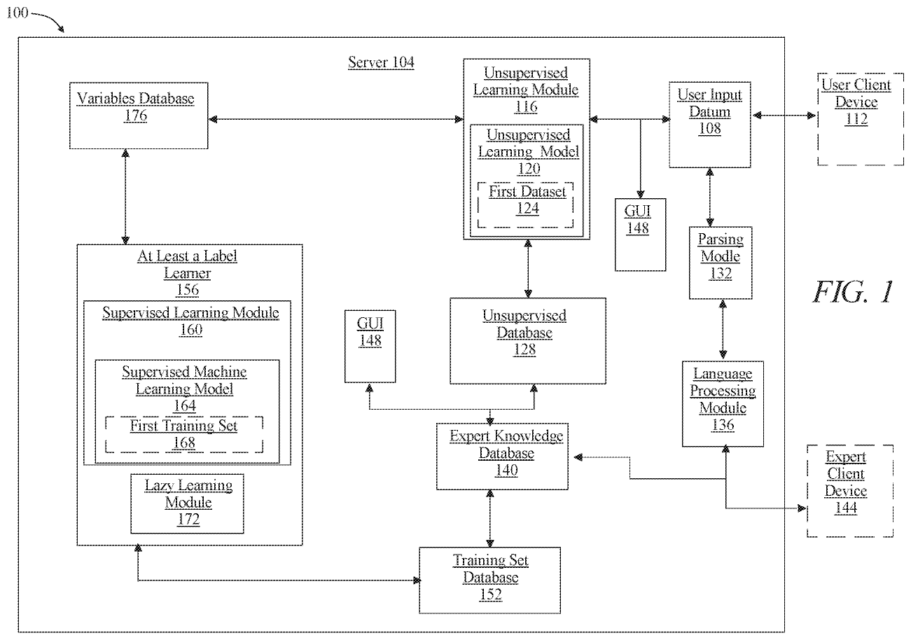

Turning now to FIG. 1, a system 100 for relating user inputs to antidote labels using artificial intelligence is illustrated. System 100 includes at least a server 104. At least a server 104 may include any computing device as described herein, including without limitation a microcontroller, microprocessor, digital signal processor (DSP) and/or system on a chip (SoC) as described herein. At least a server 104 may be housed with, may be incorporated in, or may incorporate one or more sensors of at least a sensor. Computing device may include, be included in, and/or communicate with a mobile device such as a mobile telephone or smartphone. At least a server 104 may include a single computing device operating independently, or may include two or more computing device operating in concert, in parallel, sequentially or the like; two or more computing devices may be included together in a single computing device or in two or more computing devices. At least a server 104 with one or more additional devices as described below in further detail via a network interface device. Network interface device may be utilized for connecting a at least a server 104 to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network may employ a wired and/or a wireless mode of communication. In general, any network topology may be used. Information (e.g., data, software etc.) may be communicated to and/or from a computer and/or a computing device. At least a server 104 may include but is not limited to, for example, a at least a server 104 or cluster of computing devices in a first location and a second computing device or cluster of computing devices in a second location. At least a server 104 may include one or more computing devices dedicated to data storage, security, distribution of traffic for load balancing, and the like. At least a server 104 may distribute one or more computing tasks as described below across a plurality of computing devices of computing device, which may operate in parallel, in series, redundantly, or in any other manner used for distribution of tasks or memory between computing devices. At least a server 104 may be implemented using a “shared nothing” architecture in which data is cached at the worker, in an embodiment, this may enable scalability of system 100 and/or computing device.

With continued reference to FIG. 1, at least a server 104 is configured to receive a least a user input datum 108 wherein the at least a user input datum further comprises at least a structure entry. User input datum, as used herein, includes any element of user health data. User health data may include a user complaint about a particular symptom a user may be experiencing. For example, user health data may include a description of gastrointestinal symptoms a user may be experiencing such as nausea, cramping, and diarrhea. User health datum may include a description of a previous diagnosis that a user may have received from a medical practitioner such as a functional medicine doctor. For example, user health datum may include a description of a user's allergy to tree nuts. In yet another non-limiting example, user health datum may include a user's self-reported allergy or intolerance such as an intolerance to dairy products or an elimination of food products containing gluten. User input datum includes at least a user structure entry. Structure entry as used herein, includes any entry describing any element of data relating to a user's body, body system, and/or organ contained within user's body. Structure entry may include a complaint about nonspecific pain that a user may be experiencing in user's knee. Structure entry may include a previous functional medicine test a user had performed relating to a particular body part such as a blood test utilized to analyze liver function or a salivary test analyzing hormone levels secreted by the adrenal gland. Structure entry may include a description of a certain area of user's body where user may be examining a medical condition or complaint. User input datum may include a tissue sample analysis. Tissue sample as used herein, includes any material extracted from a human body including bodily fluids and tissue. Material extracted from human body may include for example, blood, urine, sputum, fecal, and solid tissue such as bone or muscle. Tissue sample analysis as used herein, includes any tissue sample analyzed by a laboratory or medical professional such as a medical doctor for examination. In an embodiment, tissue sample analysis may include comparisons of tissue sample examination as compared to reference ranges of normal values or normal findings. For example, tissue sample analysis may include a report identifying strains of bacteria located within a user's gut examined from a stool sample and a comparison of the results to normal levels. In yet another non-limiting example, tissue sample analysis may include a report identifying hormone levels of a pre-menopausal female examined from a saliva sample. In yet another non-limiting example, tissue sample analysis may include reported results from a buccal swab that examined genetic mutations of particular genes. In yet another non-limiting example, tissue sample analysis may include a finger-prick blood test that may identify intracellular and extracellular levels of particular nutrients such as Vitamin D, Vitamin C, and Coenzyme Q10. User input datum may include a user complaint. User complaint, as used herein, includes any description of a symptom or problem that user may be experiencing. For example, user complaint may include a description of an acute onset of pain with urination that user may be experiencing or a chronic pain on user's right side user may be experiencing after eating fatty meals. In yet another non-limiting example, user complaint may include a description of constant sneezing attacks user may experience when walking outdoors.

With continued reference to FIG. 1, a user client device 112 may include, without limitation, a display in communication with at least a server 104; display may include any display as described herein. A user client device 112 may include an additional computing device, such as a mobile device, laptop, desktop computer, or the like; as a non-limiting example, the user client device 112 may be a computer and/or workstation operated by a medical professional. Output may be displayed on at least a user client device 112 using an output graphical user interface, as described in more detail below. Transmission to a user client device 112 may include any of the transmission methodologies as described herein.

With continued reference to FIG. 1, at least a server 104 is designed and configured to create at least an unsupervised machine-learning model as a function of the at least a user input datum 108 wherein creating at least an unsupervised machine-learning model further comprises selecting at least a dataset as a function of the at least a user structure entry wherein the at least a dataset further comprises at least a datum of structure entry data and at least a correlated antidote element and generating at least an unsupervised machine-learning model wherein generating the at least an unsupervised machine-learning model further comprises generating at least a clustering model to output at least a probing element containing at least a commonality label as a function of the at least a user structure entry and the at least a dataset. Unsupervised processes may, as a non-limiting example, be executed by an unsupervised learning module 116 executing on at least a server 104 and/or on another computing device in communication with at least a server 104, which may include any hardware or software module. An unsupervised machine-learning process, as used herein, is a process that derives inferences in datasets without regard to labels; as a result, an unsupervised machine-learning process may be free to discover any structure, relationship, and/or correlation provided in the data. For instance, and without limitation, unsupervised machine-learning module and/or at least a server 104 may perform an unsupervised machine-learning process on a first data set, which may cluster data of first data set according to detected relationships between elements of the first data set, including without limitation correlations of elements of user input datums to each other and correlations of antidotes to each other; such relations may then be combined with supervised machine-learning results to add new criteria for at supervised machine-learning processes as described in more detail below. As a non-limiting, illustrative example, an unsupervised process may determine that a first user input datum 108 correlates closely with a second user input datum, where the first element has been linked via supervised learning processes to a given antidote, but the second has not; for instance, the second user input datum 108 may not have been defined as an input for the supervised learning process, or may pertain to a domain outside of a domain limitation for the supervised learning process. Continuing the example a close correlation between first user input datum 108 and second user input datum 108 may indicate that the second user input datum 108 is also a good predictor for the antidote; second user input datum 108 may be included in a new supervised process to derive a relationship or may be used as a synonym or proxy for the first user input datum.

With continued reference to FIG. 1, a first probing element, as used herein, includes at least a dataset correlated to at least a user input datum and/or at least an antidote. Dataset, as used herein, includes any data and/or cohort of data that is related to at least a user input datum, where related indicates a relationship to at least a user input datum. Commonality label, as used herein, includes any suggested data and/or cluster of data that may be utilized as training data to create at least a supervised machine-learning model. Commonality label may identify certain datasets and/or cluster of datasets generated by clustering unsupervised machine-learning model that may be used as input and output pairs or labeled training data that contain associations of inputs containing input datums correlated to outputs containing antidotes that may be useful in generating supervised machine-learning algorithms. For instance and without limitation, at least a user input datum 108 containing a description of a user symptom may be utilized with at least an unsupervised machine-learning model to generate at least a first probing element containing that contains data and/or cohorts of data containing commonality labels that contain the same symptom of a user or treatment information that a user with the same symptom may have performed. In yet another non-limiting example, at least a user input data containing a tissue sample analysis of specific biomarkers within the body may be utilized in combination with at least an unsupervised machine-learning model to generate at least a first probing element containing at least a commonality label that contains data containing other users that may have had the same tissue sample analysis performed or may have had results and/or readings of specific biomarkers from a tissue sample analysis. In such an instance, first probing element may contain data describing other users that may have used particular treatment methods to alleviate a particular symptom contained within a user input datum 108 or may have used a particular treatment due to a particular tissue sample analysis or biomarker levels.

With continued reference to FIG. 1, at least a server 104 and/or unsupervised machine-learning module may detect further significant categories of user input datums, relationships of such categories to first probing elements, categories of commonality labels and/or categories of first probing elements using machine-learning processes, including without limitation unsupervised machine-learning processes as described above; such newly identified categories, as well as categories entered by experts in free-form fields, may be added to pre-populated lists of categories, lists used to identify language elements for language processing module, and/or lists used to identify and/or score categories detected in documents, as described in more detail below. In an embodiment, as additional data is added to system 100, at least a server 104 and/or unsupervised machine-learning module may continuously or iteratively perform unsupervised machine-learning processes to detect relationships between different elements of the added and/or overall data; in an embodiment, this may enable system 100 to use detected relationships to discover new correlations between biomarkers, body dimensions, tissue data, medical test data, sensor data, training set components and/or compatible substance label 120 and one or more elements of data in large bodies of data, such as genomic, proteomic, and/or microbiome-related data, enabling future supervised learning and/or lazy learning processes as described in further detail below to identify relationships between, e.g., particular clusters of genetic alleles and particular antidotes and/or suitable antidotes. Use of unsupervised learning may greatly enhance the accuracy and detail with which system 100 may generate antidotes using supervised machine-learning models as described in more detail below.

With continued reference to FIG. 1, unsupervised processes may be subjected to domain limitations. For instance, and without limitation, an unsupervised process may be performed regarding a comprehensive set of data regarding one person, such as a comprehensive medical history, set of test results, and/or classified biomarker data such as genomic, proteomic, and/or other data concerning that persons. A medical history document may include a case study, such as a case study published in a medical journal or written up by an expert. A medical history document may contain data describing and/or described by a prognosis; for instance, the medical history document may list a diagnosis that a medical practitioner made concerning the patient, a finding that the patient is at risk for a given condition and/or evinces some precursor state for the condition, or the like. A medical history document may contain data describing and/or described by a particular treatment for instance, the medical history document may list a therapy, recommendation, or other treatment process that a medical practitioner described or recommended to a patient. A medical history document may describe an outcome; for instance, medical history document may describe an improvement in a condition describing or described by a prognosis, and/or may describe that the condition did not improve. As another non-limiting example, an unsupervised process may be performed on data concerning a particular cohort of persons; cohort may include, without limitation, a demographic group such as a group of people having a shared age range, ethnic background, nationality, sex, and/or gender. Cohort may include, without limitation, a group of people having a shared value for an element and/or category of user input datum, a group of people having a shared value for an element and/or category of antidote; as illustrative examples, cohort could include all people having a certain level or range of levels of blood triglycerides, all people diagnosed with a genetic single nucleotide polymorphism, all people experiencing the same symptom or cluster of symptoms, all people with a SRD5A2 gene mutation, or the like. Persons skilled in the art, upon reviewing the entirety of this disclosure, will be aware of a multiplicity of ways in which cohorts and/or other sets of data may be defined and/or limited for a particular unsupervised learning process.

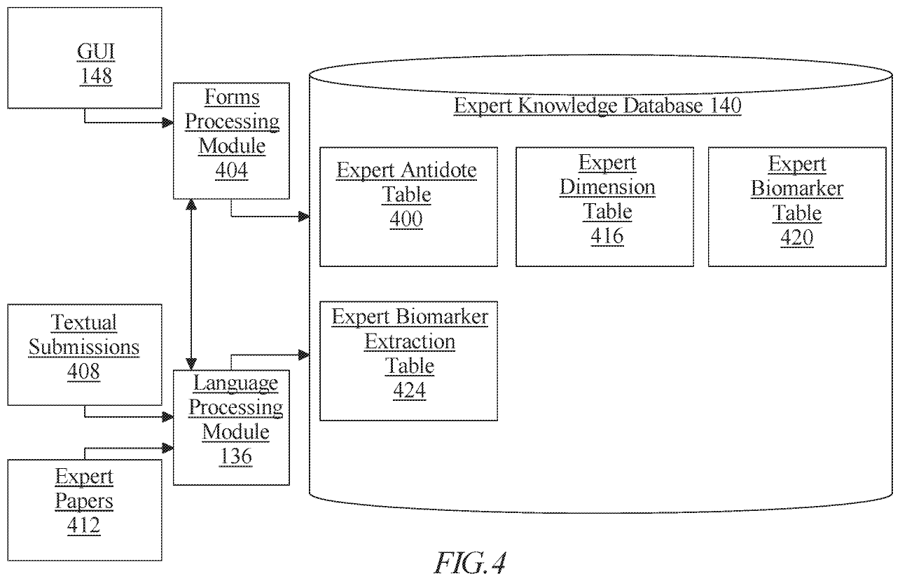

With continued reference to FIG. 1, at least a server may contain an unsupervised database 128 that may contain data utilized by at least a server 104 and/or unsupervised machine-learning module to select datasets utilized to generate at least a first probing element. In an embodiment, data contained within unsupervised database 128 may be categorized by expert inputs as described in more detail below in reference to FIG. 4. In an embodiment, at least a server and/or unsupervised machine-learning module may select at least a dataset from unsupervised database 128 to create at least an unsupervised machine-learning model as a function of matching the at least a user structure entry to at least a dataset correlated to the at least a user structure entry. In an embodiment, datasets contained within unsupervised database 128 may be organized by structures whereby a structure entry relating to a particular body part, body system, and/or organ located within the body may be matched to a dataset that is organized according to a particular body part, body system and/or organ. In an embodiment, at least a dataset may be selected as a function of matching the at least a user input datum 108 to at least a dataset containing the same user input datum 108 or matching user demographic information such as age, sex, or race. Dataset contained within unsupervised database 128 may include at least a datum of structure entry data and at least a correlated antidote element.

With continued reference to FIG. 1, system 100 may include at least a parsing module 132 operating on the at least a server. Parsing module 132 may parse the at least a user input for at least a keyword and select at least a dataset as function of the at least a keyword. Parsing module 132 may select at least a dataset by extracting one or more keywords containing words, phrases, test results, numerical scores, and the like from the at least a user input datum 108 and analyze the one or more keywords utilizing for example, language processing module as described in more detail below. Parsing module 132 may be configured to normalize one or more words or phrases of user input, where normalization signifies a process whereby one or more words or phrases are modified to match corrected or canonical forms; for instance, misspelled words may be modified to correctly spelled versions, words with alternative spellings may be converted to spellings adhering to a selected standard, such as American or British spellings, capitalizations and apostrophes may be corrected, and the like; this may be performed by reference to one or more “dictionary” data structures listing correct spellings and/or common misspellings and/or alternative spellings, or the like. Parsing module 132 may perform algorithms and calculations when analyzing tissue sample analysis and numerical test results. For instance and without limitation, parsing module 132 may perform algorithms that may compare test results contained within at least a user input datum, tissue analysis results, and/or biomarker levels to normal reference ranges or values. For example, parsing module 132 may perform calculations that determine how many standard deviations from normal levels a salvia hormone test containing salivary levels of progesterone are from normal reference ranges. In yet another non-limiting example, parsing module 132 may perform calculations between different values contained within user input datum. For example, parsing module 132 may calculate a ratio of progesterone to estradiol levels from a blood test containing a hormone panel that may include progesterone, estradiol, estrone, estriol, and testosterone serum levels.

With continued reference to FIG. 1, parsing module 132 may extract and/or analyze one or more words or phrases by performing dependency parsing processes; a dependency parsing process may be a process whereby parsing module 132 recognizes a sentence or clause and assigns a syntactic structure to the sentence or clause. Dependency parsing may include searching for or detecting syntactic elements such as subjects, objects, predicates or other verb-based syntactic structures, common phrases, nouns, adverbs, adjectives, and the like; such detected syntactic structures may be related to each other using a data structure and/or arrangement of data corresponding, as a non-limiting example, to a sentence diagram, parse tree, or similar representation of syntactic structure. Parsing module 132 may be configured, as part of dependency parsing, to generate a plurality of representations of syntactic structure, such as a plurality of parse trees, and select a correct representation from the plurality; this may be performed, without limitation, by use of syntactic disambiguation parsing algorithms such as, without limitation, Cocke-Kasami-Younger (CKY), Earley algorithm or Chart parsing algorithms. Disambiguation may alternatively or additionally be performed by comparison to representations of syntactic structures of similar phrases as detected using vector similarity, by reference to machine-learning algorithms and/or modules.

With continued reference to FIG. 1, parsing module 132 may combine separately analyzed elements from at least a user input datum 108 to extract and combine at least a keyword. For example, a first test result or biomarker reading may be combined with a second test result or biomarker reading that may be generally analyzed and interpreted together. For instance and without limitation, a biomarker reading of zinc may be reading and analyzed in combination with a biomarker reading of copper as excess zinc levels can deplete copper levels. In such an instance, parsing module 132 may combine biomarker reading off zinc and biomarker reading of copper and combine both levels to create one keyword. In an embodiment, combinations of tissue sample analysis, keywords, or test results that may be interpreted together may be received from input received from experts and may be stored in an expert knowledge database 140 as described in more detail below.

With continued reference to FIG. 1, data information describing significant categories of user input datums, relationships of such categories to first probing elements, and/or relationships of such categories antidotes may be extracted from one or more documents using a language processing module. Language processing module 136 may include any hardware and/or software module. Language processing module 136 may be configured to extract, from the one or more documents, one or more words. One or more words may include, without limitation, strings of one or characters, including without limitation any sequence or sequences of letters, numbers, punctuation, diacritic marks, engineering symbols, geometric dimensioning and tolerancing (GD&T) symbols, chemical symbols and formulas, spaces, whitespace, and other symbols, including any symbols usable as textual data as described above. Textual data may be parsed into tokens, which may include a simple word (sequence of letters separated by whitespace) or more generally a sequence of characters as described previously. The term “token,” as used herein, refers to any smaller, individual groupings of text from a larger source of text; tokens may be broken up by word, pair of words, sentence, or other delimitation. These tokens may in turn be parsed in various ways. Textual data may be parsed into words or sequences of words, which may be considered words as well. Textual data may be parsed into “n-grams”, where all sequences of n consecutive characters are considered. Any or all possible sequences of tokens or words may be stored as “chains”, for example for use as a Markov chain or Hidden Markov Model.

Still referring to FIG. 1, language processing module 136 may compare extracted words to categories of user input datums recorded by at least a server 104, and/or one or more categories of first probing elements recorded by at least a server 104; such data for comparison may be entered on at least a server 104 as using expert data inputs or the like. In an embodiment, one or more categories may be enumerated, to find total count of mentions in such documents. Alternatively or additionally, language processing module 136 may operate to produce a language processing model. Language processing model may include a program automatically generated by at least a server 104 and/or language processing module 136 to produce associations between one or more words extracted from at least a document and detect associations, including without limitation mathematical associations, between such words, and/or associations of extracted words with categories of user input datums, relationships of such categories to first probing elements, and/or categories of first probing elements. Associations between language elements, where language elements include for purposes herein extracted words, categories of user input datums, relationships of such categories to first probing elements, and/or categories of first probing elements may include, without limitation, mathematical associations, including without limitation statistical correlations between any language element and any other language element and/or language elements. Statistical correlations and/or mathematical associations may include probabilistic formulas or relationships indicating, for instance, a likelihood that a given extracted word indicates a given category of user input datum, a given relationship of such categories to a first probing element, and/or a given category of a first probing element. As a further example, statistical correlations and/or mathematical associations may include probabilistic formulas or relationships indicating a positive and/or negative association between at least an extracted word and/or a given category of user input datum, a given relationship of such categories to first probing elements, and/or a given category of probing elements; positive or negative indication may include an indication that a given document is or is not indicating a category of user input datums, relationship of such category to a first probing element, and/or category of probing elements is or is not significant. For instance, and without limitation, a negative indication may be determined from a phrase such as “Risk for lactose intolerance was not found to be correlated to age” whereas a positive indication may be determined from a phrase such as “Risk for lactose intolerance was found to be correlated to race” as an illustrative example; whether a phrase, sentence, word, or other textual element in a document or corpus of documents constitutes a positive or negative indicator may be determined, in an embodiment, by mathematical associations between detected words, comparisons to phrases and/or words indicating positive and/or negative indicators that are stored in memory by at least a server 104, or the like.

Still referring to FIG. 1, language processing module 136 and/or at least a server 104 may generate the language processing model by any suitable method, including without limitation a natural language processing classification algorithm; language processing model may include a natural language process classification model that enumerates and/or derives statistical relationships between input term and output terms. Algorithm to generate language processing model may include a stochastic gradient descent algorithm, which may include a method that iteratively optimizes an objective function, such as an objective function representing a statistical estimation of relationships between terms, including relationships between input terms and output terms, in the form of a sum of relationships to be estimated. In an alternative or additional approach, sequential tokens may be modeled as chains, serving as the observations in a Hidden Markov Model (HMM). HMMs as used herein, are statistical models with inference algorithms that that may be applied to the models. In such models, a hidden state to be estimated may include an association between an extracted word category of user input datum, a given relationship of such categories to probing element, and/or a given category of probing elements. There may be a finite number of category of user input datums a given relationship of such categories to a first probing element, and/or a given category of probing elements to which an extracted word may pertain; an HMM inference algorithm, such as the forward-backward algorithm or the Viterbi algorithm, may be used to estimate the most likely discrete state given a word or sequence of words. Language processing module 136 may combine two or more approaches. For instance, and without limitation, machine-learning program may use a combination of Naive-Bayes (NB), Stochastic Gradient Descent (SGD), and parameter grid-searching classification techniques; the result may include a classification algorithm that returns ranked associations.

Continuing to refer to FIG. 1, generating language processing model may include generating a vector space, which may be a collection of vectors, defined as a set of mathematical objects that can be added together under an operation of addition following properties of associativity, commutativity, existence of an identity element, and existence of an inverse element for each vector, and can be multiplied by scalar values under an operation of scalar multiplication compatible with field multiplication, and that has an identity element is distributive with respect to vector addition, and is distributive with respect to field addition. Each vector in an n-dimensional vector space may be represented by an n-tuple of numerical values. Each unique extracted word and/or language element as described above may be represented by a vector of the vector space. In an embodiment, each unique extracted and/or other language element may be represented by a dimension of vector space; as a non-limiting example, each element of a vector may include a number representing an enumeration of co-occurrences of the word and/or language element represented by the vector with another word and/or language element. Vectors may be normalized, scaled according to relative frequencies of appearance and/or file sizes. In an embodiment associating language elements to one another as described above may include computing a degree of vector similarity between a vector representing each language element and a vector representing another language element; vector similarity may be measured according to any norm for proximity and/or similarity of two vectors, including without limitation cosine similarity, which measures the similarity of two vectors by evaluating the cosine of the angle between the vectors, which can be computed using a dot product of the two vectors divided by the lengths of the two vectors. Degree of similarity may include any other geometric measure of distance between vectors.

Still referring to FIG. 1, language processing module 136 may use a corpus of documents to generate associations between language elements in a language processing module 136 and at least a server 104 may then use such associations to analyze words extracted from one or more documents and determine that the one or more documents indicate significance of a category of user input datums, a given relationship of such categories to probing elements, and/or a given category of probing elements. In an embodiment, at least a server 104 may perform this analysis using a selected set of significant documents, such as documents identified by one or more experts as representing good science, good clinical analysis, or the like; experts may identify or enter such documents via graphical user interface, or may communicate identities of significant documents according to any other suitable method of electronic communication, or by providing such identity to other persons who may enter such identifications into at least a server 104. Documents may be entered into at least a server 104 by being uploaded by an expert or other persons using, without limitation, file transfer protocol (FTP) or other suitable methods for transmission and/or upload of documents; alternatively or additionally, where a document is identified by a citation, a uniform resource identifier (URI), uniform resource locator (URL) or other datum permitting unambiguous identification of the document, at least a server 104 may automatically obtain the document using such an identifier, for instance by submitting a request to a database or compendium of documents such as JSTOR as provided by Ithaka Harbors, Inc. of New York.

With continued reference to FIG. 1, at least a server may include an expert knowledge database 140. Expert knowledge database 140 may include data entries reflecting one or more expert submissions of data such as may have been submitted according to any process, including without limitation by using graphical user interface. Information contained within expert knowledge database 140 may be received from input from expert client device 144. Expert client device 144 may include any information suitable for use as user client device 112 as described above. Expert knowledge database 140 may include one or more fields generated by language processing module, such as without limitation fields extracted from one or more documents as described above. For instance, and without limitation, one or more categories of user input datums and/or related probing elements and/or categories of probing elements associated with an element of user input datum 108 as described above may be stored in generalized from in an expert knowledge database 140 and linked to, entered in, or associated with entries in a user input datum. Documents may be stored and/or retrieved by at least a server 104 and/or language processing module 136 in and/or from a document database. Documents in document database may be linked to and/or retrieved using document identifiers such as URI and/or URL data, citation data, or the like; persons skilled in the art, upon reviewing the entirety of this disclosure, will be aware of various ways in which documents may be indexed and retrieved according to citation, subject matter, author, date, or the like as consistent with this disclosure.

Still referring to FIG. 1, at least a server 104 may receive a list of significant categories of user input datums and/or probing elements according to any suitable process; for instance, and without limitation, at least a server 104 may receive the list of significant categories from at least an expert. In an embodiment, at least a server 104 may provide a graphical user interface, which may include without limitation a form or other graphical element having data entry fields, wherein one or more experts, including without limitation clinical and/or scientific experts, may enter information describing one or more categories of biomarker data that the experts consider to be significant or useful for detection of conditions; fields in graphical user interface 148 may provide options describing previously identified categories, which may include a comprehensive or near-comprehensive list of types of user input datums detectable using known or recorded testing methods, for instance in “drop-down” lists, where experts may be able to select one or more entries to indicate their usefulness and/or significance in the opinion of the experts. Fields may include free-form entry fields such as text-entry fields where an expert may be able to type or otherwise enter text, enabling expert to propose or suggest categories not currently recorded. Graphical user interface 148 or the like may include fields corresponding to correlated probing elements and/or antidotes, where experts may enter data describing probing elements and/or antidotes the experts consider related to entered categories of user input datums; for instance, such fields may include drop-down lists or other pre-populated data entry fields listing currently recorded user input datums, and which may be comprehensive, permitting each expert to select a probing element and/or an antidote the expert believes to be predicted and/or associated with each category of user input datums selected by the expert. Fields for entry of probing elements and/or antidotes may include free-form data entry fields such as text entry fields; as described above, examiners may enter data not presented in pre-populated data fields in the free-form data entry fields. Alternatively or additionally, fields for entry of probing elements and/or antidotes may enable an expert to select and/or enter information describing or linked to a category of user input datums that the expert considers significant, where significance may indicate likely impact on longevity, mortality, quality of life, or the like as described in further detail below. Graphical user interface 148 may provide an expert with a field in which to indicate a reference to a document describing significant categories of user input datums, relationships of such categories to probing elements, and/or significant categories of antidotes. Any data described above may alternatively or additionally be received from experts similarly organized in paper form, which may be captured and entered into data in a similar way, or in a textual form such as a portable document file (PDF) with examiner entries, or the like.

With continued reference to FIG. 1, at least a server 104 selects at least a first training set as a function of the at least a user structure entry and the at least a first probing element containing the at least a commonality label. Training data, as used herein, is data containing correlation that a machine-learning process may use to model relationships between two or more categories of data elements. For instance, and without limitation, training data may include a plurality of data entries, each entry representing a set of data elements that were recorded, received, and/or generated together; data elements may be correlated by shared existence in a given data entry, by proximity in a given data entry, or the like. Multiple data entries in training data may evince one or more trends in correlations between categories of data elements; for instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data according to various correlations; correlations may indicate causative and/or predictive links between categories of data elements, which may be modeled as relationships such as mathematical relationships by machine-learning processes as described in further detail below. Training data may be formatted and/or organized by categories of data elements, for instance by associating data elements with one or more descriptors corresponding to categories of data elements. As a non-limiting example, training data may include data entered in standardized forms by persons or processes, such that entry of a given data element in a given field in a form may be mapped to one or more descriptors of categories. Elements in training data may be linked to descriptors of categories by tags, tokens, or other data elements; for instance, and without limitation, training data may be provided in fixed-length formats, formats linking positions of data to categories such as comma-separated value (CSV) formats and/or self-describing formats such as extensible markup language (XML), enabling processes or devices to detect categories of data.

Alternatively or additionally, and still referring to FIG. 1, training data may include one or more elements that are not categorized; that is, training data may not be formatted or contain descriptors for some elements of data. Machine-learning algorithms and/or other processes may sort training data according to one or more categorizations using, for instance, natural language processing algorithms, tokenization, detection of correlated values in raw data and the like; categories may be generated using correlation and/or other processing algorithms. As a non-limiting example, in a corpus of text, phrases making up a number “n” of compound words, such as nouns modified by other nouns, may be identified according to a statistically significant prevalence of n-grams containing such words in a particular order; such an n-gram may be categorized as an element of language such as a “word” to be tracked similarly to single words, generating a new category as a result of statistical analysis. Similarly, in a data entry including some textual data, a person's name and/or a description of a medical condition or therapy may be identified by reference to a list, dictionary, or other compendium of terms, permitting ad-hoc categorization by machine-learning algorithms, and/or automated association of data in the data entry with descriptors or into a given format. The ability to categorize data entries automatedly may enable the same training data to be made applicable for two or more distinct machine-learning algorithms as described in further detail below.

With continued reference to FIG. 1, at least a server 104 may select at least a training set from training set database. First training set may include a plurality of first data entries, each first data entry of the first training set including at least an element of structure data containing the at least a commonality label and at least a first correlated antidote label. In an embodiment, first training set may include a plurality of first data entries, each first data entry of the first training set including at least an element of input data containing the at least a commonality label and at least a first correlated antidote label. Training set database 152 may contain training sets pertaining to different categories and classification of information, including training set components which may contain sub-categories of different training sets. In an embodiment, at least a server may select at least a training set by classifying the at least a user input datum 108 to generate at least a classified user input datum 108 containing at least a body dimension label, and select at least a training set as a function of the at least a body dimension label. Body dimension as used herein, includes particular root cause pillars of disease. Dimension of the human body may include epigenetics, gut wall, microbiome, nutrients, genetics, and metabolism. In an embodiment, training set database 152 may contain training sets classified to body dimensions. In such an instance, training sets may be classified to more than one body dimension. For instance and without limitation, a training set may be classified to gut wall and microbiome. In yet another non-limiting example, a training set may be classified to nutrients and metabolism.

With continued reference to FIG. 1, epigenetic dimension, as used herein, includes any change to a genome that does not involve corresponding changes in nucleotide sequence. Epigenetic dimension may include data describing any heritable phenotypic. Phenotype, as used herein, include any observable trait of a user including morphology, physical form, and structure. Phenotype may include a user's biochemical and physiological properties, behavior, and products of behavior. Behavioral phenotypes may include cognitive, personality, and behavior patterns. This may include effects on cellular and physiological phenotypic traits that may occur due to external or environmental factors. For example, DNA methylation and histone modification may alter phenotypic expression of genes without altering underlying DNA sequence. Epigenetic dimension may include data describing one or more states of methylation of genetic material.

With continued reference to FIG. 1, gut wall dimension, as used herein includes any data describing gut wall function, gut wall integrity, gut wall strength, gut wall absorption, gut wall permeability, intestinal absorption, gut wall barrier function, gut wall absorption of bacteria, gut wall malabsorption, gut wall gastrointestinal imbalances and the like.

With continued reference to FIG. 1, microbiome dimension, as used herein, includes ecological community of commensal, symbiotic, and pathogenic microorganisms that reside on or within any of a number of human tissues and biofluids. For example, human tissues and biofluids may include the skin, mammary glands, placenta, seminal fluid, uterus, vagina, ovarian follicles, lung, saliva, oral mucosa, conjunctiva, biliary, and gastrointestinal tracts. Microbiome may include for example, bacteria, archaea, protists, fungi, and viruses. Microbiome may include commensal organisms that exist within a human being without causing harm or disease. Microbiome may include organisms that are not harmful but rather harm the human when they produce toxic metabolites such as trimethylamine. Microbiome may include pathogenic organisms that cause host damage through virulence factors such as producing toxic by-products. Microbiome may include populations of microbes such as bacteria and yeasts that may inhabit the skin and mucosal surfaces in various parts of the body. Bacteria may include for example Firmicutes species, Bacteroidetes species, Proteobacteria species, Verrumicrobia species, Actinobacteria species, Fusobacteria species, Cyanobacteria species and the like. Archaea may include methanogens such as Methanobrevibacter smithii and Methanosphaera stadtmanae. Fungi may include Candida species and Malassezia species. Viruses may include bacteriophages. Microbiome species may vary in different locations throughout the body. For example, the genitourinary system may contain a high prevalence of Lactobacillus species while the gastrointestinal tract may contain a high prevalence of Bifidobacterium species while the lung may contain a high prevalence of Streptococcus and Staphylococcus species.

With continued reference to FIG. 1, nutrient dimension as used herein, includes any substance required by the human body to function. Nutrients may include carbohydrates, protein, lipids, vitamins, minerals, antioxidants, fatty acids, amino acids, and the like. Nutrients may include for example vitamins such as thiamine, riboflavin, niacin, pantothenic acid, pyridoxine, biotin, folate, cobalamin, Vitamin C, Vitamin A, Vitamin D, Vitamin E, and Vitamin K. Nutrients may include for example minerals such as sodium, chloride, potassium, calcium, phosphorous, magnesium, sulfur, iron, zinc, iodine, selenium, copper, manganese, fluoride, chromium, molybdenum, nickel, aluminum, silicon, vanadium, arsenic, and boron. Nutrients may include extracellular nutrients that are free floating in blood and exist outside of cells. Extracellular nutrients may be located in serum. Nutrients may include intracellular nutrients which may be absorbed by cells including white blood cells and red blood cells.

With continued reference to FIG. 1, genetic dimension as used herein, includes any inherited trait. Inherited traits may include genetic material contained with DNA including for example, nucleotides. Nucleotides include adenine (A), cytosine (C), guanine (G), and thymine (T). Genetic information may be contained within the specific sequence of an individual's nucleotides and sequence throughout a gene or DNA chain. Genetics may include how a particular genetic sequence may contribute to a tendency to develop a certain disease such as cancer or Alzheimer's disease.

With continued reference to FIG. 1, metabolic dimension, as used herein, includes any process that converts food and nutrition into energy. Metabolic dimension may include biochemical processes that occur within the body.

With continued reference to FIG. 1, at least a server 104 may select at least a first training set by filtering at least a training set as a function of the at least a commonality label and selecting at least a first training set containing at least a data entry correlated to the at least a commonality label. For instance and without limitation, commonality label may include suggested clusters of data and/or datasets that may identify suggested data and/or clusters of data that may be utilized as training data. At least a server 104 may utilize commonality label to filter training sets contained within training set database 152 to exclude training sets that are not related to suggested training sets contained within commonality label. At least a server 104 may utilize commonality label to select training sets contained within training set database 152 that are related to suggested training sets and/or that contain input and output pair labels on datasets correlating to clusters generated by unsupervised machine-learning process.

With continued reference to FIG. 1, system 100 includes at least a label learner 156 operating on the at least a server 104. At least a label learner 156 is designed and configured to create at least a supervised machine-learning model using the at least a training data set, wherein the at least a supervised machine-learning model relates the at least a user input datum 108 to at least an antidote. A machine-learning process is a process that automatedly uses a body of data known as “training data” and/or a “training set” to generate an algorithm that will be performed by a computing device/module to produce outputs given data provided as inputs; this is in contrast to a non-machine-learning software program where the commands to be executed are determined in advance by a user and written in a programming language. Antidote, as used herein includes any treatment, medication, supplement, nourishment, nutrition instruction, supplement instruction, remedy, dietary advice, recommended food, recommended meal plan, or the like that may remedy at least a user input datum. For instance and without limitation, at least a user input datum 108 containing a user complaint of a stomach cramp on the right side may be utilized in combination with system 100 to select an antidote that remedies or heals user's stomach cramp. In yet another non-limiting example, at least a user input datum 108 that contains a user complaint of experiencing symptoms such as gas, diarrhea, and bloating after introducing a new food on an elimination diet may be utilized in combination with system 100 to select an antidote that eliminates other trigger foods that may also contribute to user's symptoms.

With continued reference to FIG. 1, at least a label learner 156 generates at least an antidote output using the at least a user input datum 108 and the at least a supervised machine-learning model. Supervised machine-learning models may include without limitation model developed using linear regression models. Linear regression models may include ordinary least squares regression, which aims to minimize the square of the difference between predicted outcomes and actual outcomes according to an appropriate norm for measuring such a difference (e.g. a vector-space distance norm); coefficients of the resulting linear equation may be modified to improve minimization. Linear regression models may include ridge regression methods, where the function to be minimized includes the least-squares function plus term multiplying the square of each coefficient by a scalar amount to penalize large coefficients. Linear regression models may include least absolute shrinkage and selection operator (LASSO) models, in which ridge regression is combined with multiplying the least-squares term by a factor of 1 divided by double the number of samples. Linear regression models may include a multi-task lasso model wherein the norm applied in the least-squares term of the lasso model is the Frobenius norm amounting to the square root of the sum of squares of all terms. Linear regression models may include the elastic net model, a multi-task elastic net model, a least angle regression model, a LARS lasso model, an orthogonal matching pursuit model, a Bayesian regression model, a logistic regression model, a stochastic gradient descent model, a perceptron model, a passive aggressive algorithm, a robustness regression model, a Huber regression model, or any other suitable model that may occur to persons skilled in the art upon reviewing the entirety of this disclosure. Linear regression models may be generalized in an embodiment to polynomial regression models, whereby a polynomial equation (e.g. a quadratic, cubic or higher-order equation) providing a best predicted output/actual output fit is sought; similar methods to those described above may be applied to minimize error functions, as will be apparent to persons skilled in the art upon reviewing the entirety of this disclosure.

Supervised machine-learning algorithms may include without limitation, linear discriminant analysis. Machine-learning algorithm may include quadratic discriminate analysis. Machine-learning algorithms may include kernel ridge regression. Machine-learning algorithms may include support vector machines, including without limitation support vector classification-based regression processes. Machine-learning algorithms may include stochastic gradient descent algorithms, including classification and regression algorithms based on stochastic gradient descent. Machine-learning algorithms may include nearest neighbors' algorithms. Machine-learning algorithms may include Gaussian processes such as Gaussian Process Regression. Machine-learning algorithms may include cross-decomposition algorithms, including partial least squares and/or canonical correlation analysis. Machine-learning algorithms may include naïve Bayes methods. Machine-learning algorithms may include algorithms based on decision trees, such as decision tree classification or regression algorithms. Machine-learning algorithms may include ensemble methods such as bagging meta-estimator, forest of randomized tress, AdaBoost, gradient tree boosting, and/or voting classifier methods. Machine-learning algorithms may include neural net algorithms, including convolutional neural net processes.

With continued reference to FIG. 1, supervised machine-learning algorithms may include using alternatively or additional artificial intelligence methods, including without limitation by creating an artificial neural network, such as a convolutional neural network comprising an input layer of nodes, one or more intermediate layers, and an output layer of nodes. Connections between nodes may be created via the process of “training” the network, in which elements from a training dataset are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning. This network may be trained using any training set as described herein; the trained network may then be used to apply detected relationships between elements of user input datums and antidotes.

With continued reference to FIG. 1, system 100 may include a supervised machine-learning module 160 operating on the at least a server 104 and/or on another computing device in communication with at least a server 104, which may include any hardware or software module. Supervised machine-learning algorithms, as defined herein, include algorithms that receive a training set 168 relating a number of inputs to a number of outputs, and seek to find one or more mathematical relations relating inputs to outputs, where each of the one or more mathematical relations is optimal according to some criterion specified to the algorithm using some scoring function. For instance, a supervised learning algorithm may use elements of user input datums as inputs, antidotes as outputs, and a scoring function representing a desired form of relationship to be detected between elements of user input datums and antidotes; scoring function may, for instance, seek to maximize the probability that a given element of user input datum 108 and/or combination of elements of user input datum 108 is associated with a given antidote and/or combination of antidotes to minimize the probability that a given element of user input datum 108 and/or combination of elements of user input datums is not associated with a given antidote and/or combination of antidotes. In yet another non-limiting example, a supervised learning algorithm may use elements of tissue data analysis as inputs, antidotes as outputs, and a scoring function representing a desired form of relationship to be detected between elements of tissue data analysis and antidotes. In yet another non-limiting example, a supervised learning algorithm may use elements of medical test data as inputs, antidotes as outputs, and a scoring function representing a desired form of relationship to be detected between elements of medical test data and antidotes. In yet another non-limiting example, a supervised learning algorithm may use elements of user profile information such as demographic including age, sex, race, socioeconomic status and the like; and antidotes as outputs, and a scoring function representing a desired form of relationship to be detected between elements of user profile information and antidotes. In yet another non-limiting example, a supervised learning algorithm may use elements of component categories of training data as inputs, antidotes as outputs, and a scoring function representing a desired form of relationship to be detected between elements of training data components and antidotes. Scoring function may be expressed as a risk function representing an “expected loss” of an algorithm relating inputs to outputs, where loss is computed as an error function representing a degree to which a prediction generated by the relation is incorrect when compared to a given input-output pair provided in a training set. Persons skilled in the art, upon reviewing the entirety of this disclosure, will be aware of various possible variations of supervised machine-learning algorithms that may be used to determine relation between elements of user input datums and antidotes. In an embodiment, one or more supervised machine-learning algorithms may be restricted to a particular domain for instance, a supervised machine-learning process may be performed with respect to a given set of parameters and/or categories of parameters that have been suspected to be related to a given set of user input datums, and/or are specified as linked to a medical specialty and/or field of medicine covering a particular set of symptoms, complaints, or diagnoses. As a non-limiting example, a particular set of blood test biomarkers may be typically used to recommend certain antidotes, and a supervised machine-learning process may be performed to relate those blood test biomarkers to the various antidotes; in an embodiment, domain restrictions of supervised machine-learning procedures may improve accuracy of resulting models by ignoring artifacts in training data. Domain restrictions may be suggested by experts and/or deduced from known purposes for particular evaluations and/or known tests used to evaluate antidotes. Additional supervised learning processes may be performed without domain restrictions to detect, for instance, previously unknown and/or unsuspected relationships between user input datums and antidotes.

With continued reference to FIG. 1, system 100 may include a lazy-learning module operating 172 on the at least a server 104 and/or on another computing device in communication with at least a server 104, which may include any hardware or software module. In an embodiment, at least a server 104 and/or at least a label learner 156 may be designed and configured to generate at least an antidote output by executing a lazy-learning process as a function of at least a training set and at least a user input datum. A lazy-learning process and/or protocol, which may alternatively be referred to as a “lazy loading” or “call-when-needed” process and/or protocol, may be a process whereby machine-learning is conducted upon receipt of an input to be converted to an output, by combining the input and training set to derive the algorithm to be used to produce the output on demand. For instance, an initial set of simulations may be performed to cover a “first guess” at an antidote associated with at least a user input datum, using at least a training set. As a non-limiting example, an initial heuristic may include a ranking of antidotes according to relation to a test type of at least a user input datum, one or more categories of user input datums identified in test type of at least a user input datum, and/or one or more values detected in at least a user input datum 108 sample; ranking may include, without limitation, ranking according to significance scores of associations between elements of user input datum 108 and antidotes, for instance as calculated as described above. Heuristic may include selecting some number of highest-ranking associations and/or antidote. At least a label learner 156 may alternatively or additionally implement any suitable “lazy learning” algorithm, including without limitation a K-nearest neighbors algorithm, a lazy naïve Bayes algorithm, or the like; persons skilled in the art, upon reviewing the entirety of this disclosure, will be aware of various lazy-learning algorithms that may be applied to generate antidotes as described in this disclosure, including without limitation lazy learning applications of machine-learning algorithms as described in further detail below.

With continued reference to FIG. 1, at least a server 104, and/or at least a label learner 156 may be designed and configured to generate at least an antidote output by generating a loss function of at least a user variable wherein at least a user variable further comprises at least a user treatment input and minimizes the loss function. Loss function as used herein is an expression an output of which an optimization algorithm minimizes to generate an optimal result. As a non-limiting example, at least a label learner 156 may receive and calculate variables, to calculate an output of mathematical expression using the variables, and select an antidote that produces an output having the lowest size, according to a given definition of “size,” of the set of outputs representing each of the plurality of antidotes; size may, for instance, included absolute value, numerical size, or the like. At least a user variable may include at least a user treatment input. User treatment input may include any information pertaining to a specific form of treatment that a user may prefer such as for example, a preference to select an initial antidote based on food therapy, or to select an initial antidote based on supplement therapy. Selection of different loss functions may result in identification of different antidotes as generating minimal outputs; for instance, where food therapy is associated in a first loss function with a large coefficient or weight, an antidote having a small coefficient or weight for food therapy may minimize the first loss function, whereas a second loss function wherein food therapy has a smaller coefficient but degree of variance from supplement therapy which has a larger coefficient may produce a minimal output for a different antidote having a larger food therapy but more closely hewing to a supplement therapy.

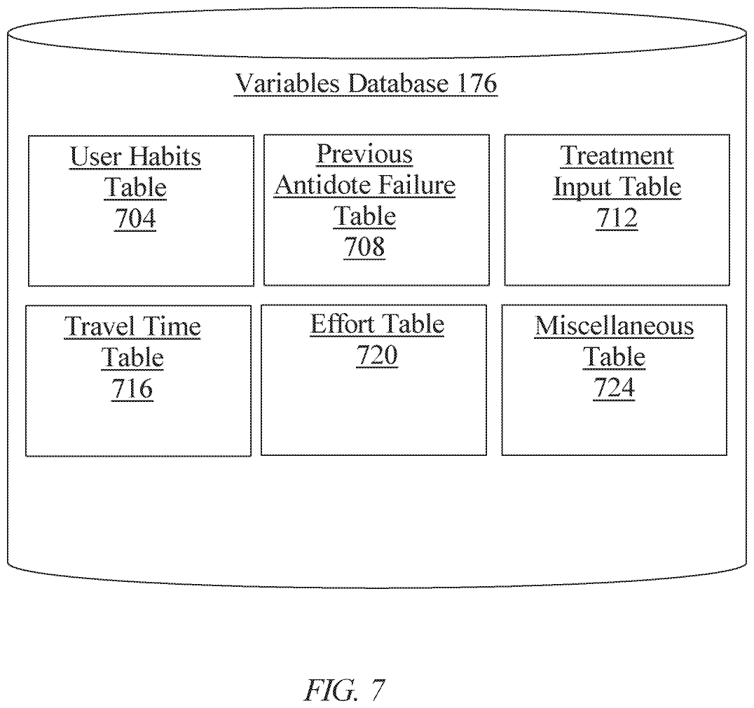

Still referring to FIG. 1, mathematical expression and/or loss function may be provided by receiving one or more user commands. For instance, and without limitation, a graphical user interface 148 may be provided to user with a set of sliders or other user inputs permitting a user to indicate relative and/or absolute importance of each variable to the user. Sliders or other inputs may be initialized prior to user entry as equal or may be set to default values based on results of any machine-learning processes or combinations thereof as described in further detail below. In an embodiment, user variables may be contained within a user variable database as described below in more detail in reference to FIG. 7.

With continued reference to FIG. 1, mathematical expression and/or loss function may be generated using machine-learning using a multi-user training set. Training set may be created using data of a cohort of persons having similar demographic, religious, health, and/or lifestyle characteristics to user. This may alternatively or additionally be used to seed a mathematical expression and/or loss function for a user, which may be modified by further machine-learning and/or regression using subsequent user selections of alimentary provision options.

With continued reference to FIG. 1, loss function analysis may measure changes in predicted values versus actual values, known as loss or error. Loss function analysis may utilize gradient descent to learn the gradient or direction that a cost analysis should take in order to reduce errors. Loss function analysis algorithms may iterate to gradually converge towards a minimum where further tweaks to the parameters produce little or zero changes in the loss or convergence by optimizing weights utilized by machine-learning algorithms. Loss function analysis may examine the cost of the difference between estimated values, to calculate the difference between hypothetical and real values. Antidotes may utilize variables to model relationships between past interactions between a user and system 100 and antidotes. In an embodiment loss function analysis may utilize variables that may impact user interactions and/or antidotes. Loss function analysis may be user specific so as to create algorithms and outputs that are customize to variables for an individual user. Variables may include any of the variables as described below in more detail in reference to FIG. 7. Variables contained within loss function analysis may be weighted and given different numerical scores. Variables may be stored and utilized to predict subsequent outputs. Outputs may seek to predict user behavior and select an optimal antidote.

With continued reference to FIG. 1, at least a server 104 may be designed and configured to receive at least a second user input datum 108 as a function of the at least an antidote output and generate at least a second antidote as a function of the at least a second user input datum. For instance and without limitation, at least a first user input datum 108 may include a user complaint of bloating symptoms and at least a first antidote may contain a recommendation for a user to eliminate a certain food or food group. In such an instance, at least a second user input datum 108 may include a user response to eliminating food suspected of causing bloating. In such an instance, second antidote may be generated using first user input datum, first antidote, and second user input datum 108 to select at least a second antidote. In such an instance, second antidote may include a recommendation to eliminate a second food or second food group if user still has persistent symptoms. If user symptoms have diminished after eliminating food contained within first antidote, then second antidote may contain a recommend to re-introduce first eliminated food after a certain quantity of time.

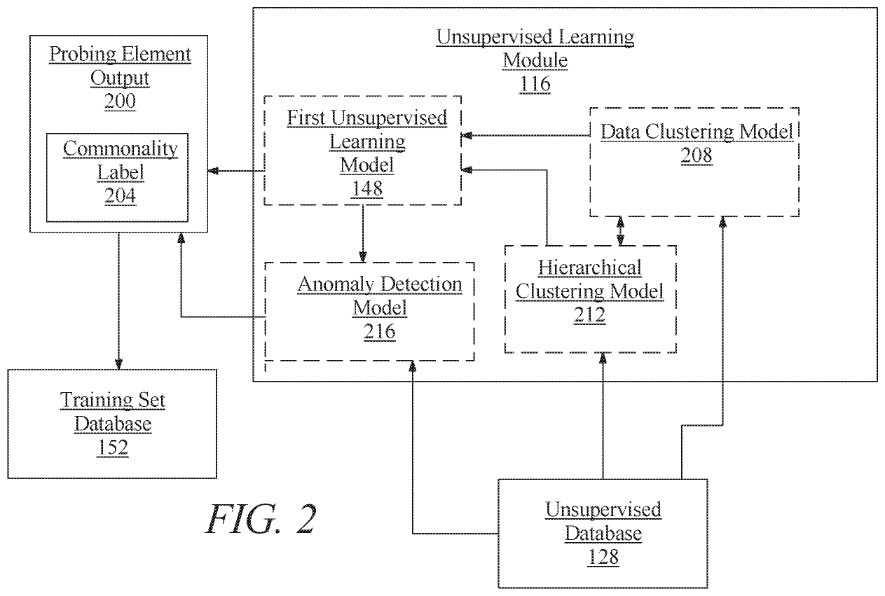

Referring now to FIG. 2, an exemplary embodiment of unsupervised learning module 116 is illustrated. Unsupervised learning may include any of the unsupervised learning processes as described herein. Unsupervised learning module 116 may create an unsupervised machine-learning model 148 that includes generating at least a clustering model to output 200 at least a probing element containing at least a commonality label 204 as a function of the at least a user structure entry and the at least a dataset. Probing element may include any of the probing elements as described herein. Commonality label may include any of the commonality labels as described herein. Dataset correlated to at least a user input datum 108 may be contained within unsupervised database 128. Unsupervised database 128 may include data describing different users and populations categorized into categories having shared characteristics as described below in more detail in reference to FIG. 1. Probing element output 200 and/or commonality label 204 may be utilized by at least a server to select at least a training set to be utilized by supervised learning module 160. Training sets may be stored and contained within training set database 152. Probing element output 200 and/or commonality label 204 generated by unsupervised learning module 116 may be utilized to select at least a first training set from training set database 152.

With continued reference to FIG. 2, unsupervised learning module 116 may include a data clustering model 208. Data clustering model 208 may group and/or segment datasets with shared attributes to extrapolate algorithmic relationships. Data clustering model 208 may group data to create clusters that may be categorized by certain classifications and/or commonality labels. Data clustering model 208 may identify commonalities in data and react based on the presence or absence of such commonalities and thereby generate commonality labels to identify clusters of data relating to probing element output 200. For instance and without limitation, data clustering model 208 may identify other data that contains matching symptoms to a user input datum 108 or other data that contains a treatment option to a symptom contained within a user input datum. Data clustering model 208 may utilize other forms of data clustering algorithms including for example, hierarchical clustering, k-means, mixture models, OPTICS algorithm, and DBSCAN.

With continued reference to FIG. 2, unsupervised learning module 116 may include a hierarchical clustering model 212. Hierarchical clustering model 212 may group and/or segment datasets into hierarchy clusters including both agglomerative and divisive clusters. Agglomerative clusters may include a bottom up approach where each observation starts in its own cluster and pairs of clusters are merged as one moves up the hierarchy. Divisive clusters may include a top down approach where all observations may start in one cluster and splits are performed recursively as one moves down the hierarchy.

With continued reference to FIG. 2, unsupervised learning module 116 may include an anomaly detection model 216. Anomaly detection model 216 may include identification of rare items, events or observations that differ significant from the majority of the data. Anomaly detection model 216 may function to observe and find outliers. For instance and without limitation, anomaly detect may find and examine data outliers such as user symptoms that did not respond to treatment or that resolved spontaneously without medical intervention.

With continued reference to FIG. 2, unsupervised learning module 116 may include a plurality of other models that may perform other unsupervised machine-learning processes. This may include for example, neural networks, autoencoders, deep belief nets, Hebbian learning, adversarial networks, self-organizing maps, expectation-maximization algorithm, method of moments, blind signal separation techniques, principal component analysis, independent component analysis, non-negative matrix factorization, singular value decomposition (not pictured).

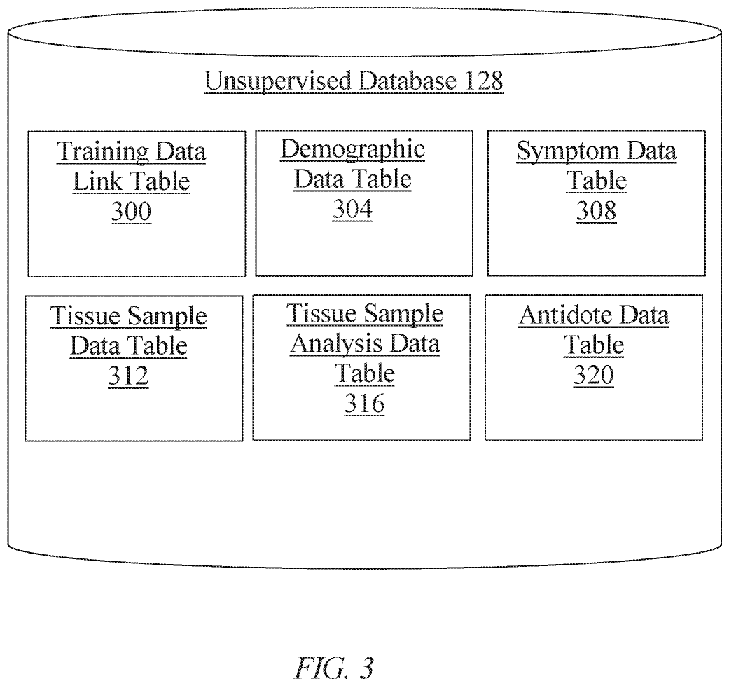

Referring now to FIG. 3, an exemplary embodiment of unsupervised database 128 is illustrated, which may be implemented as a hardware or software module. Unsupervised database 128 may be implemented, without limitation, as a relational database, a key-value retrieval datastore such as a NOSQL database, or any other format or structure for use as a datastore that a person skilled in the art would recognize as suitable upon review of the entirety of this disclosure. Unsupervised database 128 may contain data that may be utilized by unsupervised module 116 to find trends, cohorts, and shared datasets between data contained within unsupervised database 128 and at least a user input datum. In an embodiment, data contained within unsupervised database 128 may be categorized and/or organized according to shared characteristics. For instance and without limitation, one or more tables contained within unsupervised database 128 may include training data link table 300; training data link table 300 may contain information linking data sets contained within unsupervised database 128 to datasets contained within training set database 152. For example, dataset contained within unsupervised database 128 may also be contained within training set database 152, which may be linked through training data link table 300. In yet another non-limiting example, training data link table 300 may contain information linking data sets contained within unsupervised database 128 to datasets contained within training set database 152 such as when dataset and training set may include data sourced from the same user or same cohort of users. For instance and without limitation, one or more tables contained within unsupervised database 128 may include demographic table 304; demographic table 304 may include datasets pertaining to demographic information. Demographic information may include datasets describing age, sex, ethnicity, socioeconomic status, education level, marital status, income level, religion, offspring information, and the like. For instance and without limitation, one or more tables contained within unsupervised database 128 may include symptom data table 308; symptom data table may include datasets describing symptoms. Symptom data table 308 may include symptoms a user may experience such as for example acute symptoms or ongoing chronic symptoms describing a particular condition or disease state. For instance and without limitation, one or more tables contained within unsupervised database 128 may include tissue sample data table 312; tissue sample data table 312 may include datasets pertaining to tissue samples. Tissue sample data table 312 may include data describing results from a bone marrow biopsy or a saliva test. For instance and without limitation, one or more tables within unsupervised database 128 may include tissue sample analysis data table 316; tissue sample analysis data table 316 may include datasets describing one or more tissue sample analysis results. For example, tissue sample analysis data table 316 may include datasets describing test results and associated analysis from a blood test examining intracellular levels of nutrients and how intracellular levels of nutrient compare to normal values. For instance and without limitation, one or more tables within unsupervised database 128 may include antidote data table 320; antidote data table 320 may include datasets describing one or more antidotes. For example, antidote data table 320 may include a particular treatment utilized which may be linked to a particular diagnosis or symptom. One or more database tables contained within unsupervised database table 128 may include a diagnosis table, a chief complaint table, a structure entry table, a geographic table, (not pictured). Persons skilled in the art will be aware of the various database tables that may be contained within unsupervised database table 128 consistently within the purview of this disclosure.

Referring now to FIG. 4, an exemplary embodiment of expert knowledge database 140 is illustrated. Expert knowledge database 140 may include any data structure for ordered storage and retrieval of data, which may be implemented as a hardware or software module, and which may be implemented as any database structure suitable for use as unsupervised database 128. One or more database tables in expert knowledge database 140 may include, as a non-limiting example, an expert antidote table 400. Expert antidote table 400 may be a table relating user input datums as described above to expert antidotes; for instance, where an expert has entered data relating a user input datum 108 such as symptoms including runny nose, sneezing, and coughing to an antidote such as hot tea and an herbal supplement, one or more rows recording such an entry may be inserted in expert antidote table 400. In an embodiment, a forms processing module 404 may sort data entered in a submission via graphical user interface 148 by, for instance, sorting data from entries in the graphical user interface 148 to related categories of data; for instance, data entered in an entry relating in the graphical user interface 148 to an antidote may be sorted into variables and/or data structures for storage of antidotes, while data entered in an entry relating to a user input datum 108 and/or an element thereof may be sorted into variables and/or data structures for the storage of, respectively, categories of user input datums or elements of user input datums. Where data is chosen by an expert from pre-selected entries such as drop-down lists, data may be stored directly; where data is entered in textual form, language processing module 136 may be used to map data to an appropriate existing label, for instance using a vector similarity test or other synonym-sensitive language processing test to map classified biometric data to an existing label. Alternatively or additionally, when a language processing algorithm, such as vector similarity comparison, indicates that an entry is not a synonym of an existing label, language processing module 136 may indicate that entry should be treated as relating to a new label; this may be determined by, e.g., comparison to a threshold number of cosine similarity and/or other geometric measures of vector similarity of the entered text to a nearest existent label, and determination that a degree of similarity falls below the threshold number and/or a degree of dissimilarity falls above the threshold number. Data from expert textual submissions 408, such as accomplished by filling out a paper or PDF form and/or submitting narrative information, may likewise be processed using language processing module 136. Data may be extracted from expert papers 412, which may include without limitation publications in medical and/or scientific journals, by language processing module 136 via any suitable process as described herein. Persons skilled in the art, upon reviewing the entirety of this disclosure, will be aware of various additional methods whereby novel terms may be separated from already-classified terms and/or synonyms therefore, as consistent with this disclosure. Expert antidote table 400 may include a single table and/or a plurality of tables; plurality of tables may include tables for particular categories of antidotes such as a pharmaceutical intervention antidote, a food intervention antidote, a supplement intervention antidote, a fitness intervention antidote (not shown), to name a few non-limiting examples presented for illustrative purposes only.

With continued reference to FIG. 4, one or more database tables in expert knowledge database 140 may include, an expert dimension table 416 may list one or more body dimensions as described by experts, and one or more biomarkers associated with one or more body dimensions. Body dimensions may include any of the body dimensions as described herein, including for example an epigenetic dimension, a gut wall dimension, a genetic dimension, a microbiome dimension, a nutrient dimension, a metabolic dimension, and the like. As a further example an expert biomarker table 420 may list one or more biomarkers as described and input by experts and associated dimensions that biomarkers may be classified into. For instance and without limitation, an expert biomarker table 420 may include one or more tables detailing biomarkers commonly associated with a particular body dimension such as microbiome. In yet another non-limiting example, expert biomarker table 420 may include one or more tables detailing biomarkers commonly associated with a particular diagnosis such as an elevated fasting blood glucose level and diabetes mellitus. As an additional example, an expert biomarker extraction table 424 may include information pertaining to biological extraction and/or medical test or collection necessary to obtain a particular biomarker, such as for example a tissue sample that may include a urine sample, blood sample, hair sample, cerebrospinal fluid sample, buccal sample, sputum sample, and the like. Tables presented above are presented for exemplary purposes only; persons skilled in the art will be aware of various ways in which data may be organized in expert knowledge database 140 consistently with this disclosure.

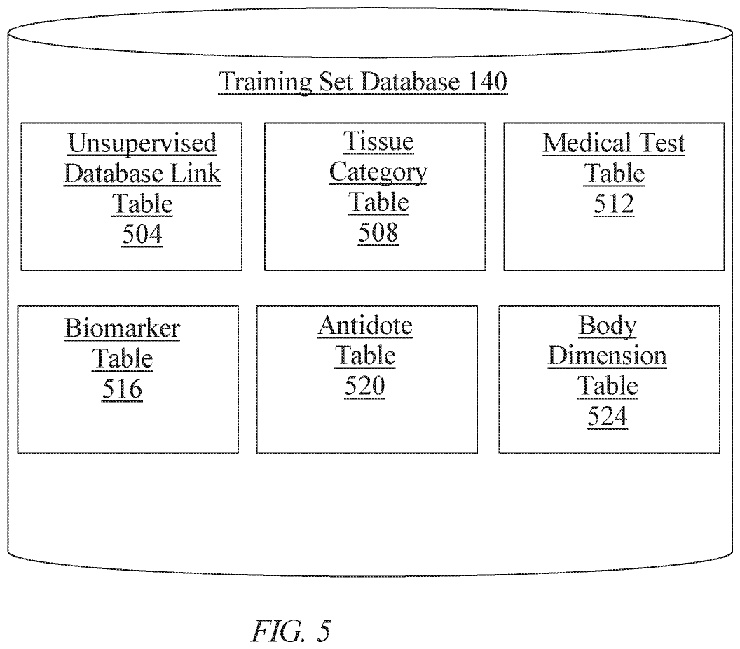

Referring now to FIG. 5, an exemplary embodiment of training set database 152 is illustrated which may include any data structure for ordered storage and retrieval of data, which may be implemented as a hardware or software module, and which may be implemented as any database structure suitable for use as unsupervised database 128. For instance and without limitation, one or more database tables contained within training set database 152 is unsupervised database link table 504; unsupervised database link table 504 may contain information linking data sets contained within training set database 152 to datasets contained within unsupervised database 128. For example, dataset contained within training set database 152 may also be contained within unsupervised database 128, which may be linked through unsupervised database link table 504. In yet another non-limiting example, database link table 504 may contain information linking data sets contained within training set database 152 to datasets contained within unsupervised database, such as when dataset and training set may include data sourced from the same user or same cohort of users. For instance and without limitation, one or more database tables contained within training set database 152 may include tissue category table 508; tissue category table 508 may contain training sets pertaining to tissue categories may contain training sets pertaining to different tissue samples that may be analyzed for biomarkers which may be correlated to one or more antidotes. Tissue may include for example blood, cerebrospinal fluid, urine, blood plasma, synovial fluid, amniotic fluid, lymph, tears, saliva, semen, aqueous humor, vaginal lubrication, bile, mucus, vitreous body, gastric acid, which may be correlated to an antidote. For instance and without limitation, one or more database tables contained within training set database 152 may include medical test table 512; medical test table 512 may include training sets pertaining to medical tests which may be correlated to one or more antidotes. Medical tests may include any medical test, medical procedure, and/or medical test or procedure results that may be utilized to obtain biomarkers and tissue samples such as for example, an endoscopy procedure utilized to collect a liver tissue sample, or a blood draw collected and analyzed for circulating hormone levels. For instance and without limitation, one or more database tables contained within training set database 152 may include biomarker table 516; biomarker table 516 may include biomarkers correlated to one or more antidotes. For example, a biomarker such as high triglycerides may be correlated to an antidote such as exercise. For instance and without limitation, one or more database tables contained within training set database 152 may include antidote table 520; antidote table 520 may include antidotes correlated to other antidotes. For example, antidote table 520 may include a first antidote that is correlated to a second antidote as part of a treatment sequence. For instance and without limitation, one or more database tables contained within training set database 152 may include body dimension table 524; body dimension table 524 may include dataset labeled to one or more body dimensions and a correlated antidote label. For example, body dimension table 524 may include data showing low microbial colonization of Saccharomyces Boulardii containing categorized to microbiome body dimension and containing a correlated antidote that includes supplementation with Saccharomyces Boulardii. Tables presented above are p148resented for exemplary purposes only; persons skilled in the art will be aware of various ways in which data may be organized in training set database 152 consistently with this disclosure.

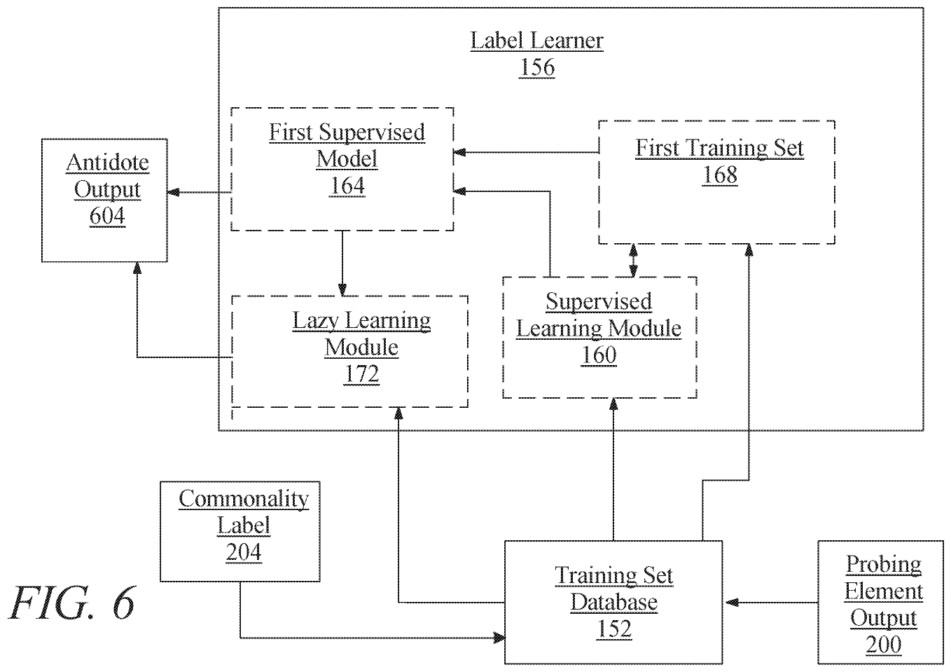

Referring now to FIG. 6, an exemplary embodiment of at least a label learner 156 is illustrated. Training set data utilized to generate first supervised model 164 may be selected from training set database 152. First training set 168 may be selected from training set database 152 as a function of user input datum 108 and probing element output 200. For instance and without limitation, probing element output 200 may be utilized to match dataset contained within probing element output to dataset contained within training set database 152. This may be done for instance, by matching dataset to find similar inputs and outputs that will be utilized in first training set 168 from inputs and outputs generated within probing element output 200. In yet another non-limiting example, matching may include matching category of data selected within probing element output 200 to a similar category of data contained within training set database 152.

With continued reference to FIG. 6, at least a label learner 156 may include supervised machine-learning module 160. Supervised machine-learning module 160 may be configured to perform any supervised machine-learning algorithm as described above in reference to FIG. 1. This may include for example, support vector machines, linear regression, logistic regression, naïve Bayes, linear discriminant analysis, decision trees, k-nearest neighbor algorithm, neural networks, and similarity learning. Supervised machine-learning module 160 may generated a first supervised machine-learning model 164 which may be utilized to generate antidote output 604. Antidote output 604 may include any treatment, medication, supplement, nourishment, nutrition instruction, supplement instruction, remedy, dietary advice, recommended food, recommended meal plan, or the like that may remedy at least a user input datum. For instance and without limitation, at least a user input datum 108 that includes a user complaint of gas and bloating after eating may be utilized in combination with label learner 156 to generate antidote output 604 that includes a treatment recommendation for gas and bloating, such as trying an elimination diet.

With continued reference to FIG. 6, at least a label learner 156 may include a lazy-learning module 172. Lazy-learning module may be configured to perform a lazy-learning algorithm, including any of the lazy-learning algorithms as described above in reference to FIG. 1. Lazy-learning algorithm may include for example, performing k-nearest neighbors and/or lazy naïve Bayes rules. In an embodiment, lazy-learning module 172 may generate a lazy-learning algorithm. In an embodiment, label learner 156 may perform supervised machine-learning algorithm and a lazy-learning algorithm together, alone, or in any combination to generate antidote output 804.

Referring now to FIG. 7, an exemplary embodiment of variables database 176 is illustrated, which may include any data structure for ordered storage and retrieval of data, which may be implemented as a hardware or software module, and which may be implemented as any database structure suitable for use as unsupervised database 128. For instance and without limitation, one or more data tables contained within variables database 176 may include habits table 704; habits table 704 may include information describing a user's daily habits, such as whether a user lives alone or with other individuals who may be able to provide support or aid a user with a particular antidote. For instance and without limitation, one or more data tables contained within variables database 176 may include previous antidote failure table 708; previous antidote failure table 708 may include information describing previous antidotes that user may have previously tried and not had any success in alleviating or eliminating a user complaint or problem. For example, previous antidote failure table 708 may include information describing a particular medication that didn't alleviate user's symptoms or a particular food that still causes user gastrointestinal distress. For instance and without limitation, one or more data tables contained within variables database 176 may include treatment input table 712; treatment input table 712 may include user preference information for a particular category of antidote or treatment. For example, treatment input table 712 may include information describing a user's preference for treatment with food or treatment with supplements. In an embodiment, treatment input table 712 may contain a hierarchy listing treatment preference in descending order of preference and importance to a particular user. For instance and without limitation, one or more data tables contained within variables database 176 may include travel timetable 716; travel timetable 716 may include information describing a user's preference to travel a particular distance to obtain a particular antidote. For example, travel timetable 716 may include information describing a user's preference to travel a maximum of twenty miles to a health food store to purchase a particular supplement. For instance and without limitation, one or more data tables contained within variables database 176 may include effort table 720; effort table 720 may include information describing a user's particular effort to any given antidote. For example, effort table 720 may include information such as a user's effort to comply with a supplement plan that requires dosing three times per day and also a user's inability to comply with a nutrition plan that requires cooking three separate meals each day. For instance and without limitation, one or more data tables contained within variables database 176 may include miscellaneous table 724; miscellaneous table 724 may include miscellaneous variables that may be weighted and utilized by at least a server 104 when generating and minimizing a loss function.

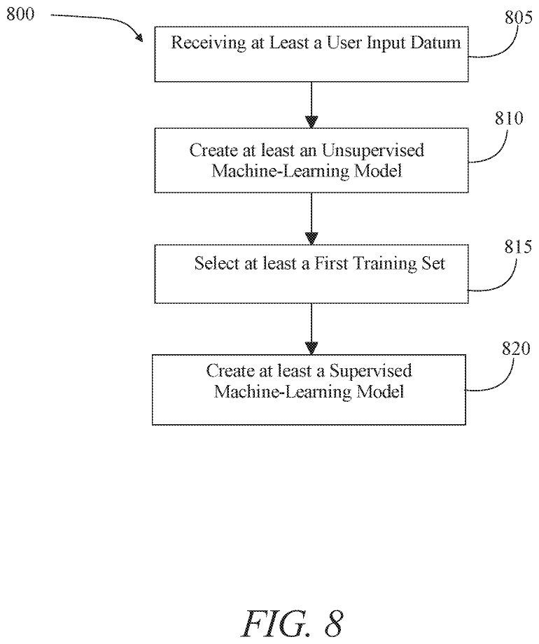

Referring now to FIG. 8, an exemplary embodiment of a method 800 of relating user inputs to antidote labels using artificial intelligence is illustrated. At step 805, at least a server receives at least a user input datum wherein the at least a user input datum further comprises at least a user structure entry. User input datum 108 may include any of the user input datums as described above in reference to FIG. 1. For example, at least a user input datum 108 may include a current symptom that a user may be experiencing such as a runny nose or ankle pain. At least a user input datum 108 may include a tissue sample analysis. Tissue sample analysis may include any of the tissue sample analysis as described herein. For example, tissue sample analysis may include a report from a saliva test that a user may have performed analyzing hormone levels of a user such as salivary levels of testosterone, progesterone, estradiol, estriol, estrone, DHEA, and cortisol. In yet another non-limiting example, tissue sample analysis may include a report from a finger-prick blood test that may have analyzed immunoglobulin g (IGG) food that a user may have a food sensitivity to. At least a user datum may include a user complaint. User complaint may include a chief complaint of a user. Chief complaint may include a description of a medical problem or issue a user may be experiencing or a symptom that might not go away. For example, user complaint may include a description of a rash that won't go away. In yet another non-limiting example, user complaint may include a previously problem that a user may have had and a treatment that did not work. User input datum may include at least a user structure entry. User structure entry may include any of the structure entries as described above in reference to FIGS. 1-8. For example, user structure entry may include a description of a particular affected area of user's body such as an inflamed big toe or a complaint of abdominal tenderness and burning. User structure entry may be related to a tissue sample analysis. For example, user structure entry may include a particular test result or bodily fluid sample that was analyzed as a function of a particular body system. For instance and without limitation, user structure entry may include a particular stool sample user analyzed as it relates to user's stomach pain or a salivary cortisol test user had performed as it relates to user's mind chatter and inability to fall asleep at night. At least a user input datum 108 may be received using any network methodology as described herein.

With continued reference to FIG. 8, at step 810 at least a server creates at least an unsupervised machine-learning model as a function of the at least a user input datum wherein creating at least an unsupervised machine-learning model further comprises selecting at least a dataset as a function of the at least a user structure entry wherein the at least a dataset further comprises at least a datum of structure entry data and at least a correlated antidote element and generating at least an unsupervised machine-learning model wherein generating at least an unsupervised machine-learning model further comprises generating at least a clustering model to output at least a probing element containing at least a commonality label as a function of the at least a user structure entry and the at least a dataset. In an embodiment, at least a server may be configured to create at least an unsupervised machine-learning model as a function of matching at least a user structure entry to at least a dataset correlated to the at least a user structure entry. In an embodiment, datasets contained within unsupervised database 128 may be organized and/or categorized based on categories of structure entries thereby allowing user entries to be matched as a function of shared categories. Unsupervised machine-learning model may include any of the unsupervised machine-learning models as described above in reference to FIGS. 1-8. Unsupervised machine-learning model may include algorithms such as clustering, hierarchical clustering, and anomaly detection as described above in more detail in reference to FIG. 2. In an embodiment, at least a server 104 may create at least an unsupervised machine-learning model as a function of selecting at least a dataset from unsupervised database 128 as a function of the at least a user structure entry. For example, at least a server 104 may select at least a dataset that may include a shared symptom or shared chief complaint contained within at least a user input datum. In an embodiment, datasets contained within unsupervised database 128 may be categorized by data elements containing shared characteristics as described above in more detail in reference to FIG. 3. In such an instance, at least a server 104 may select at least a dataset that may be categorized based on a shared trait or characteristic that may be contained within at least a user input datum. In an embodiment, at least a server 104 may parse the at least a user input datum 108 to extract at least a keyword and select at least a dataset as a function of the at least a keyword. In an embodiment, parsing module 132 may extract a keyword that may be utilized to select at least a dataset as a function of matching the keyword to a category of dataset contained within unsupervised database 128. For example, parsing module 132 may extract a keyword such as a description of a user complaint or symptom that may be utilized to match the keyword to a category of data contained within unsupervised database 128. Parsing may be performed using any of the methods as described above in reference to FIG. 1.

With continued reference to FIG. 8, unsupervised machine-learning model outputs at least a first probing element containing at least a commonality label as a function of the at least a user structure entry and the at least a dataset. First probing element may contain suggested data associations, and/or categories of data that may be utilized to select training sets. For example, first probing element may contain a dataset containing potential antidotes correlated to at least a user input datum 108 that may be utilized to select a training set that contains user input datum 108 as input and correlated antidotes as outputs. In yet another non-limiting example, first probing element may contain categories of data that may be utilized to select potential training sets such as for example by classifying data contained within unsupervised database 128. For instance and without limitation, unsupervised learning module 116 may be utilized to create at least a first probing element that contains datasets of other users with similar symptoms to those contained within user input datum. For example, at least a user input datum 108 that contains a user complaint of migraines after eating may be utilized by unsupervised machine-learning module to select datasets of other users who experience migraines after eating. Such datasets can then be utilized to create training sets that contain input and output labels that correlate to inputs consisting of users who experience migraines after eating to outputs that contain antidotes that helped other users eliminate migraines after eating. First probing element containing at least a commonality label may include suggested datasets and/or clusters of data generated by clustering model that may be utilized as training sets. Commonality label may suggest and/or contain data describing datasets that may be utilized as training sets to be utilized by at least a server 104 and/or at least a label learner when generating at least a supervised machine-learning model

With continued reference to FIG. 8, at step 815 at least a server 104 selects at least a first training set 168 as a function of the at least a user structure entry and the at least a first probing element containing the at least a commonality label. First training set 168 may be selected from training set database 152 as a function of at least a first probing element. For example, at least a first probing element may be utilized to select at least a first training set 168 that contains labels and/or categories of data contained within first probing element. For example, at least a first probing element may contain categories of clustered datasets produced from data selected from unsupervised database 128 and clustered into categories by unsupervised learning module 116 each containing commonality labels. Categories of clustered datasets produced by creating an unsupervised learning model 120 may be utilized to identify training sets that may contain the same commonality label to generate training set that will be utilized by supervised learning module 160 to generate a supervised machine-learning model 164. For example and without limitation, at least a user input datum 108 may contain a user complain of a symptom user may be experiencing such as a runny nose. Unsupervised learning module 116 may utilize datasets contained within unsupervised database 128 that may be selected as a function of user input datum 108 such as by matching user demographic information or similar complaints. Unsupervised learning module 116 may utilized data selected from supervised database 128 in combination with at least a user input datum 108 to generate clusters of groups by generating an unsupervised learning model 120 using first dataset 124. Clusters may then be contained within first probing element containing at least a commonality label to select training sets from training set database 152 that contain similar demographics, user complaints, suggested antidotes and the like, to clusters identified from unsupervised learning module 116 containing shared commonality labels. In an embodiment, datasets contained within unsupervised database 128 may be utilized as training sets by supervised learning module 160 and/or at least a label learner 156. Training sets may include at least a first element of classified data and at least a correlated second element of classified data. Classified data may include any data that has been classified such as by unsupervised learning module 116 by clustering to generate classifications. Classifications generated by unsupervised learning module 116 such as by data clustering, or hierarchical clustering may be utilized to classify data to select training sets utilized by unsupervised learning module 116. Selecting at least a training set may also be done by extracting at least a keyword from a user input datum 108 such as by parsing module 132 as described above.

With continued reference to FIG. 8, at least a first training set may be selected by filtering at least a training set as a function of the at least a commonality label and selecting at least a first training set containing at least a data entry correlated to the at least a commonality label. For instance and without limitation, at least a server 104 may filter training sets contained within training set database 152 to eliminate training sets that do not contain matching commonality labels. For example, at least a server 104 may eliminate training sets that do not contain commonality labels that match commonality labels contained within probing element. At least a server 104 may select at least a first training set correlated to the at least a commonality label. For example, commonality label may identify clusters of data that contain user input datums relating to user input data received by at least a server 104. At least a server 104 may utilize commonality label to identify datasets contained within training set database 152 to find training sets containing user input datums correlated to user input datum received by at least a server. In an embodiment, first training set may include a plurality of first data entries, each first data entry of the first training set including at least an element of structure data containing the at least a commonality label and at least a correlated first antidote label.

With continued reference to FIG. 8, at least a server 104 may select at least a first training set 168 by classifying the at least a user input datum 108 to generate at least a classified user input datum 108 containing at least a body dimension label and select at least a first training set 168 as a function of the at least a body dimension label. Body dimension may include any of the body dimensions as described herein. For example, at least a user input datum 108 containing a description of foods user cannot consume due to the presence or absence of certain bacteria in user's gastrointestinal tract may be classified by at least a server as relating to body dimension such as microbiome and may contain at least a body dimension label that contains microbiome. In such an instance, body dimension label containing microbiome may be utilized by at least a server 104 to select at least a first training set 168 from training set database 152 that may be categorized as belonging to microbiome. In an embodiment, user input datum 108 may contain more than one body dimension label that may be utilized to select more than one training set that may be utilized by supervised learning module 160 to generate a supervised machine-learning model 164.

With continued reference to FIG. 8, at step 820 at least a label learner 156 operating on the at least a server 104 creates at least a supervised machine-learning model as a function of the at least a first training set and the at least a commonality label, wherein creating the at least a supervised machine-learning model further comprises generating at least a supervised machine-learning model to output at least an antidote output as a function of relating the at least a user input datum to at least an antidote. Supervised machine-learning model 164 may include any of the supervised machine-learning models as described above in reference to FIGS. 1-8, including for example supervised machine-learning models such as support vector machines, linear regression, logistic regression, naïve Bayes, linear discriminant analysis, decision trees, k-nearest neighbor algorithms, neural networks, and/or similarity networks. Supervised machine-learning model 164 may be generated utilizing first training set 168 and the at least a user input datum 108. First training set 168 may be selected utilizing any of the methodologies as described above. At least an antidote may include any of the antidotes as described above in reference to FIGS. 1-8. At least an antidote may include for example a treatment or remedy for a user as a function of at least a user input datum. Supervised machine-learning model 164 may include any of the supervised machine-learning models as described above in reference to FIGS. 1-8. In an embodiment, at least an antidote may be generated by executing a lazy learning process as a function of the at least a first training set 168 and the at least a user input datum. Lazy-learning process may be performed by a lazy-learning module 172 operating on at least a server 104. Lazy-learning may include any of the lazy-learning processes as described above in reference to FIGS. 1-8, including for example algorithms such as k-nearest neighbors and lazy naïve Bayes rules.

With continued reference to FIG. 8, in an embodiment, generating at least an antidote may include generating a supervised machine-learning model 164 and/or lazy learning model generated by lazy learning module 172. Generating at least an antidote may include generating by at least a label learner 156 operating on at least a server 104 a loss function of at least a user variable wherein the at least a variable further comprises a treatment input and minimizing the loss function. Loss function may include any of the loss functions as described above in reference to FIG. 1. Generating loss function may include any of the methodologies as described above in reference to FIGS. 1-8. User variables may include any of the user variables as described above in reference to FIG. 7. User variable may include at least a treatment input which may include any of the user input regarding a preference for user treatment as described above in reference to FIG. 7. For example and without limitation, user treatment variable may include a preference for a user to receive a nutraceutical antidote such as a supplement as compared to a food based antidote such as a dietary change or food elimination. In an embodiment, user treatment variable may be utilized to select at least an antidote that may match user treatment preference. For example, user treatment variable indicating a preference to receive a nutraceutical treatment may be utilized to eliminate antidotes that do not contain a nutraceutical treatment and to select at least an antidote that does contain a nutraceutical treatment. In an embodiment, user treatment variable may include a hierarchy of user treatment preferences, such as for example a ranking of most preferred treatment down to least preferred treatment. In an embodiment, user treatment variable may be generated as a function of user past interactions with system 100 such as for example previous user antidotes.

With continued reference to FIG. 8, at least a server 104 is configured to receive at least a second user input datum 108 as a function of the at least an antidote output and generate at least a second antidote as a function of the at least a second user input datum. In an embodiment, second user input datum 108 may include a user response to at least an antidote such as for example, a user remark if the at least an antidote improved user's symptom. In an embodiment, second user input datum 108 may include a second tissue sample analysis with remarks describing changes from first tissue sample analysis. For example, a first tissue sample analysis such as a stool test showing low levels of commensal bacteria in a user's gastrointestinal tract may be re-evaluated such as by taking a second stool test after a user started at least an antidote containing a probiotic containing specific bacterial strains to repopulate user's gastrointestinal tract with specific strains. In such an instance, second user input datum 108 may include a second stool test analysis and at least a second antidote may be generated to determine if user needs new probiotic strains, can stop taking probiotic strains, or possibly needs a new higher dose of probiotic strains. In yet another non-limiting example, a first user input datum 108 may include a user symptom such as a complaint of bloating after eating whereby at least an antidote may be generated to recommend removal of certain foods or food groups selected from training sets and datasets of users who complained of similar symptoms. In such an instance, a second user input datum 108 may be received that may contain a description as to whether or not user's symptoms disappeared, improved, or worsened, whereby a second antidote may be generated that may include a recommendation of new foods to eliminate or new foods to reintroduce to user's diet.

It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

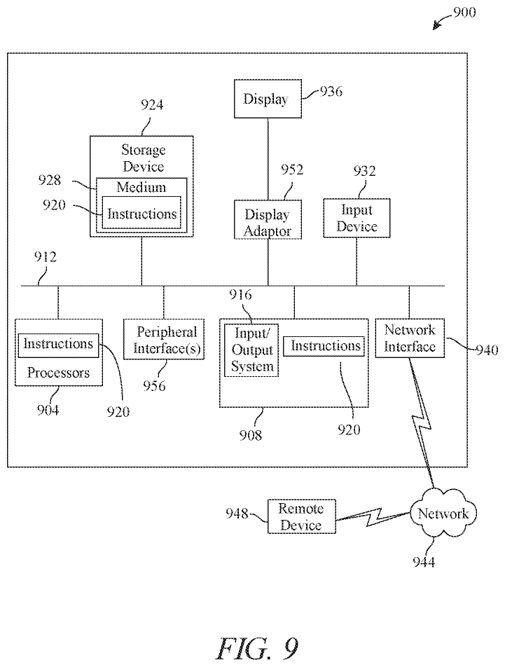

FIG. 9 shows a diagrammatic representation of one embodiment of a computing device in the exemplary form of a computer system 900 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 900 includes a processor 904 and a memory 908 that communicate with each other, and with other components, via a bus 912. Bus 912 may include any of several types of bus structures including, but not limited to, a memory bus, a memory controller, a peripheral bus, a local bus, and any combinations thereof, using any of a variety of bus architectures.

Memory 908 may include various components (e.g., machine-readable media) including, but not limited to, a random access memory component, a read only component, and any combinations thereof. In one example, a basic input/output system 916 (BIOS), including basic routines that help to transfer information between elements within computer system 900, such as during start-up, may be stored in memory 908. Memory 908 may also include (e.g., stored on one or more machine-readable media) instructions (e.g., software) 920 embodying any one or more of the aspects and/or methodologies of the present disclosure. In another example, memory 908 may further include any number of program modules including, but not limited to, an operating system, one or more application programs, other program modules, program data, and any combinations thereof.

Computer system 900 may also include a storage device 924. Examples of a storage device (e.g., storage device 924) include, but are not limited to, a hard disk drive, a magnetic disk drive, an optical disc drive in combination with an optical medium, a solid-state memory device, and any combinations thereof. Storage device 924 may be connected to bus 912 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 924 (or one or more components thereof) may be removably interfaced with computer system 900 (e.g., via an external port connector (not shown)). Particularly, storage device 924 and an associated machine-readable medium 928 may provide nonvolatile and/or volatile storage of machine-readable instructions, data structures, program modules, and/or other data for computer system 900. In one example, software 920 may reside, completely or partially, within machine-readable medium 928. In another example, software 920 may reside, completely or partially, within processor 904.

Computer system 900 may also include an input device 932. In one example, a user of computer system 900 may enter commands and/or other information into computer system 900 via input device 932. Examples of an input device 932 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 932 may be interfaced to bus 912 via any of a variety of interfaces (not shown) including, but not limited to, a serial interface, a parallel interface, a game port, a USB interface, a FIREWIRE interface, a direct interface to bus 912, and any combinations thereof. Input device 932 may include a touch screen interface that may be a part of or separate from display 936, discussed further below. Input device 932 may be utilized as a user selection device for selecting one or more graphical representations in a graphical interface as described above.

A user may also input commands and/or other information to computer system 900 via storage device 924 (e.g., a removable disk drive, a flash drive, etc.) and/or network interface device 940. A network interface device, such as network interface device 940, may be utilized for connecting computer system 900 to one or more of a variety of networks, such as network 944, and one or more remote devices 948 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 944, may employ a wired and/or a wireless mode of communication. In general, any network topology may be used. Information (e.g., data, software 920, etc.) may be communicated to and/or from computer system 900 via network interface device 940.

Computer system 900 may further include a video display adapter 952 for communicating a displayable image to a display device, such as display device 936. Examples of a display device include, but are not limited to, a liquid crystal display (LCD), a cathode ray tube (CRT), a plasma display, a light emitting diode (LED) display, and any combinations thereof. Display adapter 952 and display device 936 may be utilized in combination with processor 904 to provide graphical representations of aspects of the present disclosure. In addition to a display device, computer system 900 may include one or more other peripheral output devices including, but not limited to, an audio speaker, a printer, and any combinations thereof. Such peripheral output devices may be connected to bus 912 via a peripheral interface 956. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods, systems, and software according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

What is claimed is:

1. A system for relating user inputs to antidote labels using artificial intelligence, the system comprising:
at least a server, the at least a server designed and configured to:
receive at least a user input datum wherein the at least a user input datum further comprises at least a user structure entry;
create at least an unsupervised machine-learning model as a function of the at least a user input datum wherein creating at least an unsupervised machine-learning model further comprises:
selecting at least a dataset as a function of the at least a user structure entry wherein the at least a dataset further comprises at least a datum of structure entry data and at least a correlated antidote element; and
generating at least an unsupervised machine-learning model wherein generating the at least an unsupervised machine-learning model further comprises generating at least a clustering model to output at least a probing element containing at least a commonality label as a function of the at least a user structure entry and the at least a dataset; and
select at least a first training set as a function of the at least a user structure entry and the at least a first probing element containing the at least a commonality label; and
at least a label learner operating on the at least a server; the at least a label learner designed and configured to:
create at least a supervised machine-learning model as a function of the at least a first training set and the at least a commonality label, wherein creating the at least a supervised machine-learning model further comprises generating at least a supervised machine-learning model to output at least an antidote output as a function of relating the at least a user input datum to at least an antidote.

2. The system of claim 1, wherein the at least a server is further configured to receive at least a user input datum containing at least a tissue sample analysis.

3. The system of claim 1, wherein the at least a server is further configured to receive at least a user input datum containing at least a user complaint.

4. The system of claim 1, wherein the at least a server is further configured to create at least an unsupervised machine-learning model as a function of matching the at least a user structure entry to at least a dataset correlated to the at least a user structure entry.

5. The system of claim 1, wherein the at least a server is further configured to select at least a first training set further comprises:
filtering at least a training set as a function of the at least a commonality label; and
selecting at least a first training set containing at least a data entry correlated to the at least a commonality label.

6. The system of claim 1, wherein the at least a server is further configured to:
receive at least a user input datum;
classify the at least a user input datum to generate at least a classified user input datum containing at least a body dimension label; and
select at least a first training set as a function of the at least a body dimension label.

7. The system of claim 1, wherein the at least a first training set further comprises a plurality of first data entries, each first data entry of the first training set including at least an element of structure data containing the at least a commonality label and at least a correlated first antidote label.

8. The system of claim 1, wherein the at least a label learner is further designed and configured to generate at least an antidote output by executing a lazy learning process as a function of the at least a first training set and the at least a user input datum.

9. The system of claim 1, wherein the at least a label learner is further designed and configured to generate at least an antidote output by:
generating a loss function of at least a user variable wherein the at least a user variable further comprises a treatment input; and
minimizing the loss function.

10. The system of claim 1, wherein the at least a server is further configured to:
receive at least a second user input datum as a function of the at least an antidote output; and
generate at least a second antidote as a function of the at least a second user input datum.

11. A method of relating user inputs to antidote labels using artificial intelligence, the method comprising:
receiving by at least a server at least a user input datum wherein the at least a user input datum further comprises at least a user structure entry;
creating by the at least a server at least an unsupervised machine-learning model as a function of the at least a user input datum wherein creating at least an unsupervised machine-learning model further comprises:
selecting at least a dataset as a function of the at least a user structure entry wherein the at least a dataset further comprises at least a datum of structure entry data and at least a correlated antidote element; and
generating at least an unsupervised machine-learning model wherein generating the at least an unsupervised machine-learning model further comprises generating at least a clustering model to output at least a probing element containing at least a commonality label as a function of the at least a user structure entry and the at least a dataset;
selecting by the at least a server at least a first training set as a function of the at least a user structure entry and the at least a first probing element containing the at least a commonality label; and
creating by at least a label learner operating on the at least a server at least a supervised machine-learning model as a function of the at least a first training set and the at least a commonality label, wherein creating the at least a supervised machine-learning model further comprises generating at least a supervised machine-learning model to output at least an antidote output as a function of relating the at least a user input datum to at least an antidote.

12. The method of claim 11, wherein receiving at least a user input datum further comprises receiving at least a tissue sample analysis.

13. The method of claim 11, wherein receiving at least a user input datum further comprises receiving at least a user complaint.

14. The method of claim 11, wherein creating at least an unsupervised machine-learning model further comprises matching the at least a user structure entry to at least a dataset correlated to the at least a user structure entry.

15. The method of claim 11, wherein selecting at least a first training set further comprises:
filtering at least a training set as a function of the at least a commonality label; and
selecting at least a first training set containing at least a data entry correlated to the at least a commonality label.

16. The method of claim 11, wherein selecting at least a first training set further comprises:
receiving at least a user input datum;
classifying the at least a user input datum to generate at least a classified user input datum containing at least a body dimension label; and
selecting at least a first training set as a function of the at least a body dimension label.

17. The method of claim 11, wherein selecting at least a first training set further comprises selecting a first training set containing a plurality of first data entries, each first data entry of the first training set including at least an element of structure data containing the at least a commonality label and at least a correlated first antidote label.

18. The method of claim 11 further comprising generating at least an antidote output by:
executing a lazy learning process as a function of the at least a first training set and the at least a user input datum.

19. The method of claim 11 further comprising generating at least an antidote output by:
generating a loss function of at least a user variable wherein the at least a user variable further comprises a treatment input; and
minimizing the loss function.

20. The method of claim 11 further comprising:
receiving at least a second user input datum as a function of the at least an antidote output; and
generating at least a second antidote as a function of the at least a second user input datum.
